(19) United States
(12) Patent Application Publication
(10) Pub. No.: US 2022/0079701 A1
(43) Pub. Date: Mar. 17, 2022

(54) APPLIANCE FOR THE TRANSPORT OF BIOMEDICAL DEVICES

(71) Applicant: EUROSETS S.R.L., Medolla (IT)

(72) Inventors: Antonio PETRALIA, Medolla (IT); Nicola GHELLI, Medolla (IT); Paolo FONTANILI, Medolla (IT); Alberto PEDERZOLI, Medolla (IT)

(73) Assignee: EUROSETS S.R.L., Medolla (IT)

(21) Appl. No.: 17/422,722

(22) PCT Filed: Oct. 2, 2019
(86) PCT No.: PCT/IB2019/058389

§ 371 Date: Jul. 13, 2021

(30) Foreign Application Priority Data
Jan. 25, 2019 (IT) 102019000001143

(51) Int. Cl. A61B 50/13; A61B 50/15; B62B 5/00

(52) U.S. Cl. CPC A61B 50/13; B62B 2203/02; B62B 5/00; A61B 50/15

(57) ABSTRACT

An appliance for the transport of biomedical devices. The appliance comprises at least one load-bearing structure, movement device/unit/component/structure of the load-bearing structure with respect to a reference surface and at least one supporting top of at least one biomedical device associated with the load-bearing structure, where the load-bearing structure comprises first removable hooking device/unit/component/structure to the movement device/unit/component/structure, the first hooking device/unit/component/structure being movable between an engagement configuration, wherein the load-bearing structure is constrained to the movement device/unit/component/structure, and a release configuration wherein the load-bearing structure is released and removable from the movement device/unit/component/structure.

### TECHNICAL FIELD

The present invention relates to an appliance for the transport of biomedical devices.

### BACKGROUND ART

Appliances are known for the transport, both inside and outside hospitals, of biomedical devices, such as devices for extracorporeal blood circulation, e.g. oxygenators and centrifugal pumps, or other devices used in hospital procedures and in emergency situations.

These appliances of known type are generally of the type of trolleys or the like, i.e. they comprise a load-bearing structure associated with movement means, of the wheel type, and supporting a supporting top of one or more biomedical devices.

These appliances of known type do have a number of drawbacks.

Their use, in fact, is limited to the transport of biomedical devices only and does not permit their use, for example, on hospital beds or stretchers.

These known appliances are therefore not very flexible and require, for example, that the supported biomedical devices be removed and moved from them following the movement of the patient.

### DESCRIPTION OF THE INVENTION

The main aim of the present invention is to provide an appliance for the transport of biomedical devices which is more flexible to use than the appliances of known type.

Within this aim, one object of the present invention is to provide an appliance which permits simplifying the movement and use of biomedical devices during hospital procedures that also involve moving the patient.

Another object of the present invention is to provide an appliance for the transport of biomedical devices which allows overcoming the aforementioned drawbacks of the prior art in a simple, rational, easy, effective to use and low cost solution.

The objects set forth above are achieved by the present appliance for the transport of biomedical devices according to claim 1.

### BRIEF DESCRIPTION OF THE DRAWINGS

Other characteristics and advantages of the present invention will become more evident from the description of a preferred, but not exclusive, embodiment of an appliance for the transport of biomedical devices, illustrated by way of a non-limiting example in the accompanying tables of drawings in which:

FIG. 1 is a side elevation view of an appliance according to the invention in a first embodiment, with the first hooking means in engagement configuration with the movement means;

FIG. 2 is a front elevation view of the appliance of FIG. 1;

FIG. 3 is a side elevation view of the appliance of FIG. 1, with the second hooking means in engagement configuration with an auxiliary structure;

FIG. 4 is an enlarged view of the first hooking means of the appliance of FIG. 1;

FIG. 5 is an enlarged view of the second hooking means of the appliance of FIG. 1;

FIG. 6 is an enlarged plan view from above of the anchoring means of the appliance of FIG. 1;

FIG. 7 is a side elevation view of the anchoring means of FIG. 6 in engagement with a biomedical device;

FIG. 8 is a first axonometric view of an appliance according to the invention, in a second embodiment, with the first hooking means in engagement configuration;

FIG. 9 is an axonometric view of the appliance of FIG. 1 with the hooking means in the release configuration and the load-bearing structure separated from the movement means;

FIG. 10 is an axonometric view of the appliance of FIG. 8 supporting an appliance for the support of biomedical devices, in a first configuration of use;

FIG. 11 is an axonometric view of the appliance of FIG. 1 supporting an appliance for the transport of biomedical devices, in a second configuration of use.

### EMBODIMENTS OF THE INVENTION

With particular reference to these illustrations, reference numeral 1 globally indicates an appliance for the transport of biomedical devices.

The appliance 1 comprises at least one load-bearing structure 2, movement means 3 of the load-bearing structure 2 with respect to a reference surface S, such as e.g. the floor, and at least one supporting top 4 associated with the load-bearing structure 2 and intended to support at least one biomedical device O, P. More particularly, the load-bearing structure 2 comprises a plurality of tubular elements 5, mechanically connected to each other, e.g. by welding, threaded members or other methods known the technician in the sector.

According to the invention, the load-bearing structure 2 comprises first removable hooking means 6 to the movement means 3, wherein the first hooking means 6 are movable between an engagement configuration, wherein the load-bearing structure 2 is locked together with the movement means 3, and a release configuration wherein the load-bearing structure 2 is released and removable from the movement means 3.

The movement means 3 preferably comprise a plurality of wheels movable in rotation around relevant axes and at least one connecting frame 7 supporting the wheels 3. The load-bearing structure 2 in turn comprises one or more coupling elements 8a, 8b adapted to operate in conjunction with the connecting frame 7, wherein the first hooking means 6 secure and release the coupling elements 8a, 8b to and from the connecting frame 7 in the engagement configuration and in the release configuration, respectively.

More particularly, the connecting frame 7 also comprises a plurality of tubular elements 9 mechanically connected to each other.

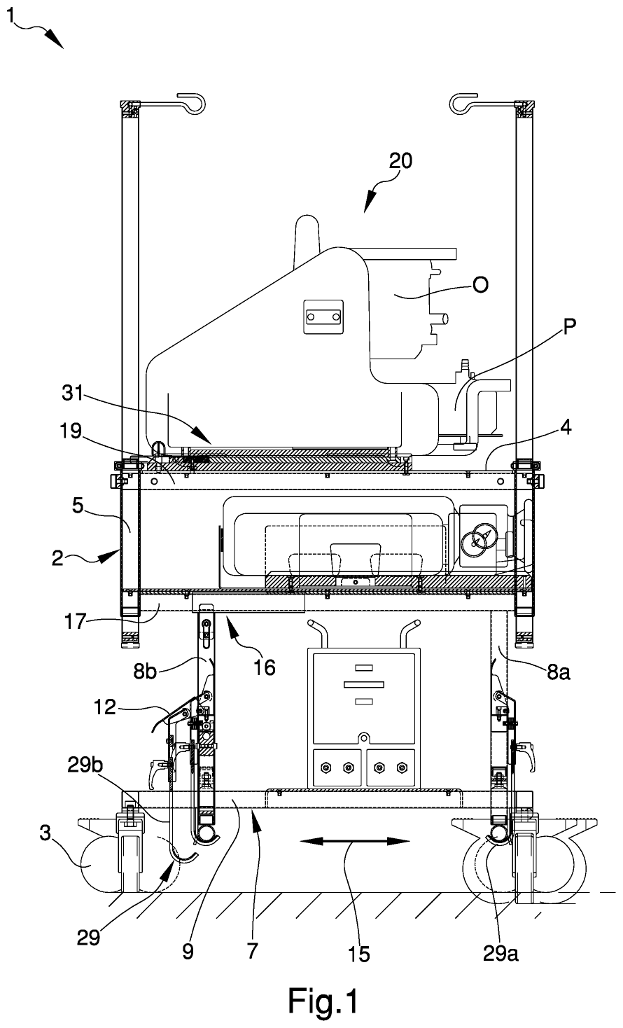

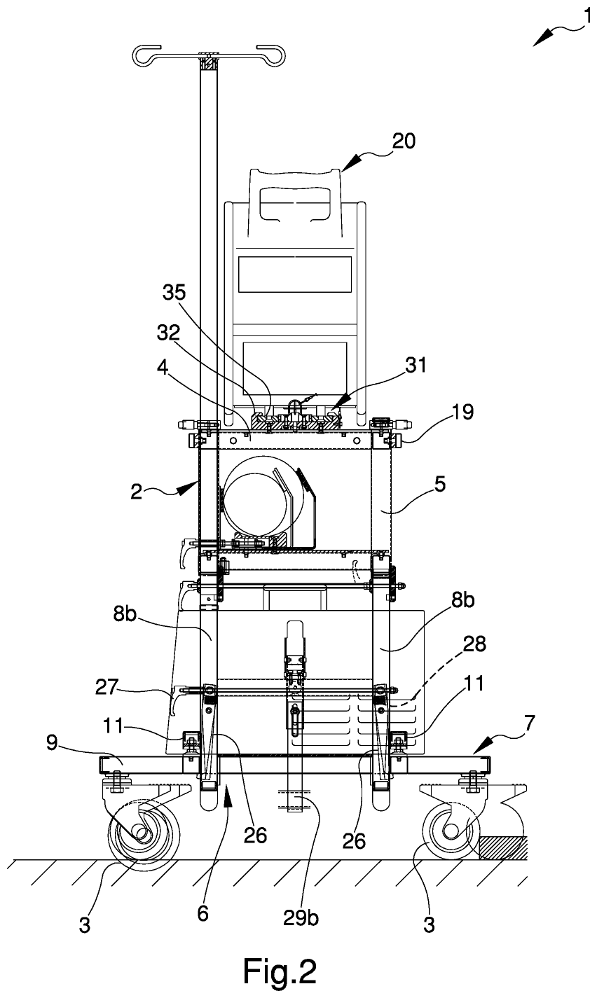

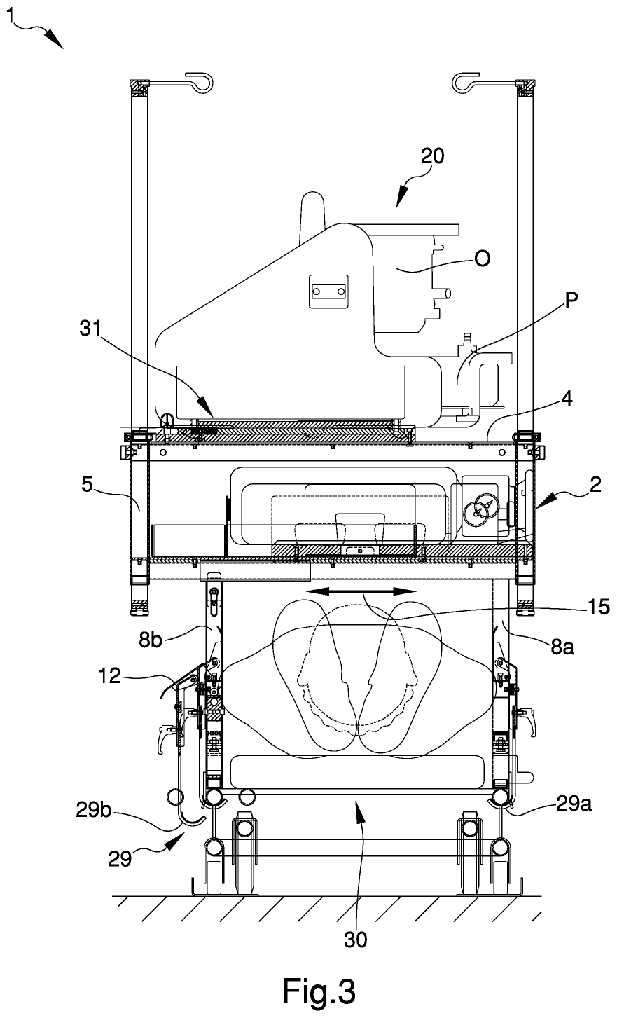

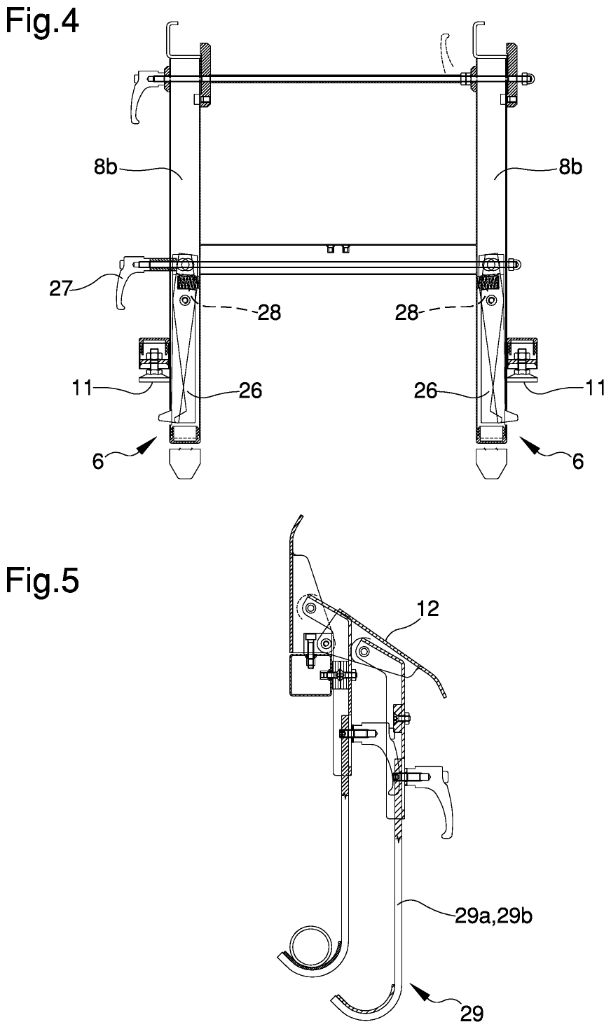

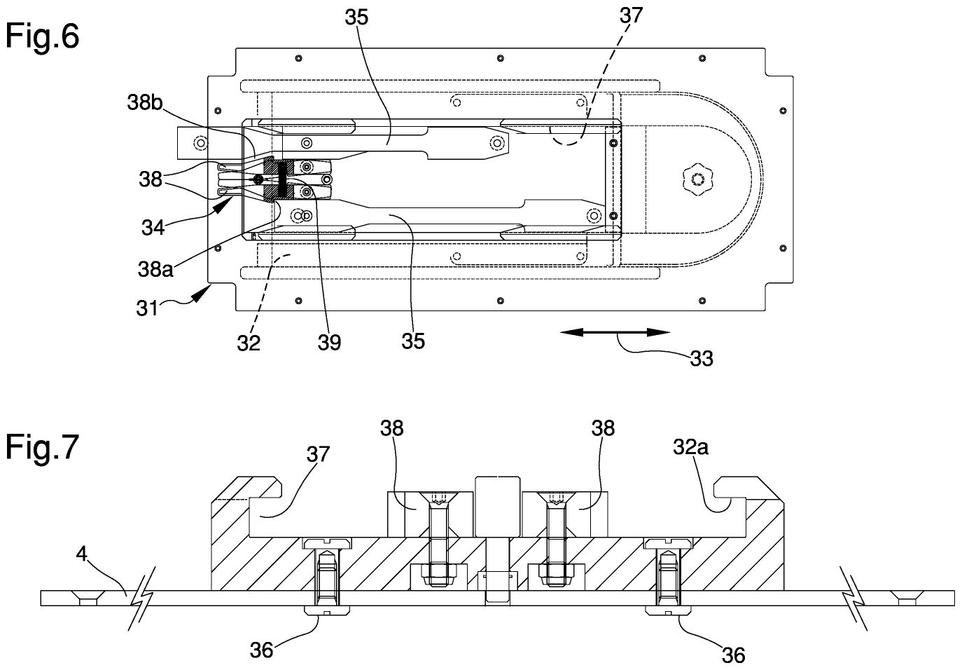

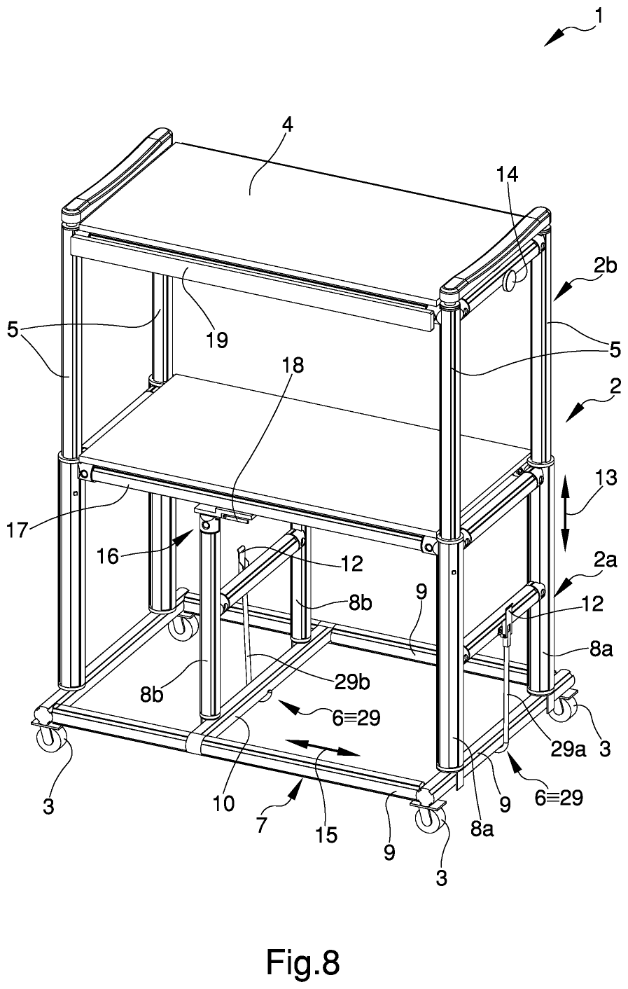

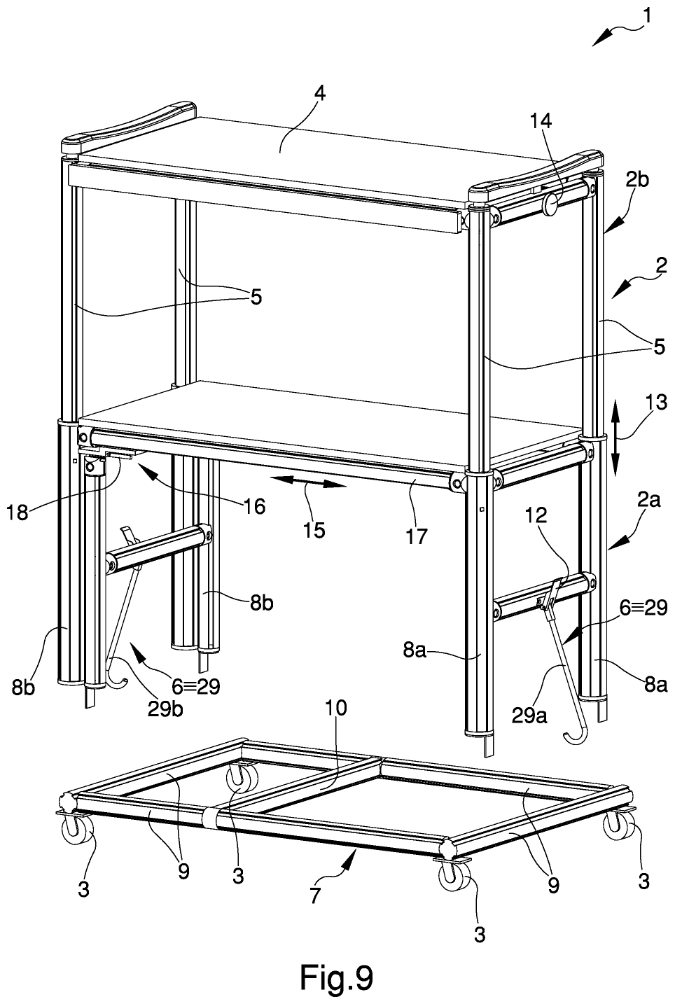

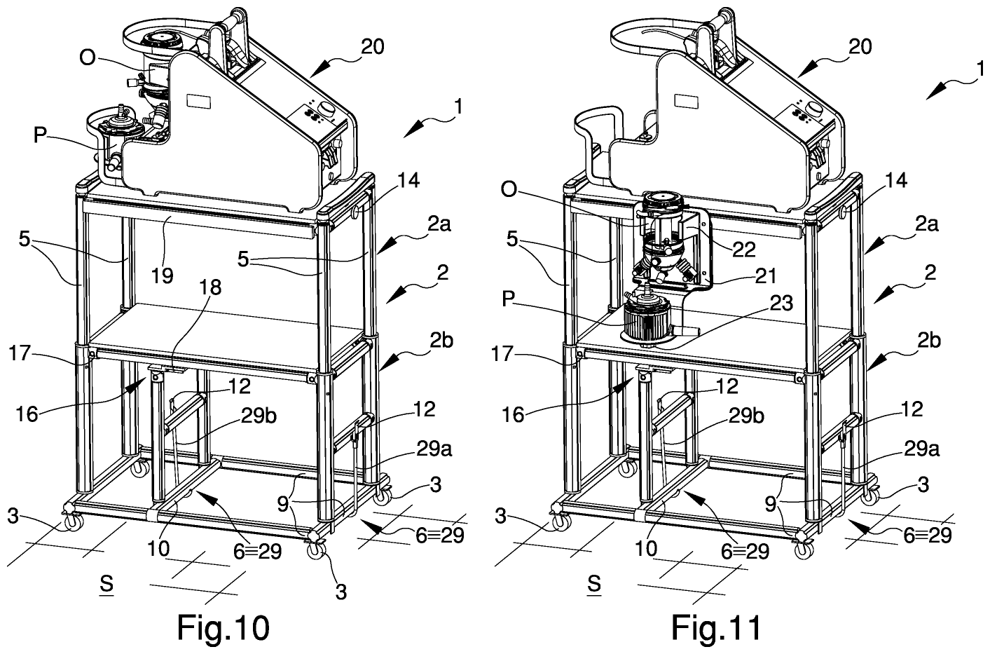

Conveniently, the coupling elements 8a, 8b comprise relevant supporting surfaces with the connecting frame 7. For example, in the first embodiment shown in FIGS. 1 to 7, the coupling elements 8a, 8b define relevant supporting feet 11 adapted to contact the connecting frame 7. In the second embodiment, shown in FIGS. 8 to 11, on the other hand, the coupling elements 8a, 8b comprise an open housing seat, substantially shaped like a reversed L, which rests on the connecting frame 7 or on another element 10 connected thereto.

Advantageously, the load-bearing structure 2 comprises at least a first and at least a second coupling element 8a and 8b arranged facing each other, i.e. having the respective housing seats mutually opposite, and movable in the direction of mutual approach/removal 15. More in detail, the load-bearing structure 2 comprises a pair of first coupling elements 8a arranged side by side and a pair of second coupling elements 8b arranged side by side.

Conveniently, the appliance 1 comprises displacement means 16 of the second coupling elements 8b along the direction of mutual approach/removal 15.

The displacement means 16 preferably comprise at least one guide element 17 associated with the load-bearing structure 2 and extending along the direction of mutual approach/removal 15 and the second coupling elements 8b are associated in a sliding manner with the guide element 17, e.g. by means of a slide connection.

In the embodiment shown in the figures, the displacement means 16 comprise two guide elements 17 arranged substantially parallel to each other and the first coupling elements 8a are fixed with respect to the guide elements 17.

Conveniently, the appliance 1 comprises second removable locking means 18 for locking the displacement of the second coupling elements 8b along the direction of mutual approach/removal 15. The second locking means 18 are e.g. of the type of a lever which can be operated manually by an operator to lock or release the second coupling elements 8b from the guide elements 17.

Advantageously, in the first embodiment shown in FIGS. 1 to 7, the first hooking means 6 comprise at least one abutment element 26 associated with at least one of the coupling elements 8a, 8b and movable with respect thereto between the engagement configuration and the release configuration. The abutment element 26 can therefore be moved with respect to the relevant coupling element 8a, 8b with which it is associated.

Preferably, at least one of the coupling elements 8a, 8b has a tubular conformation, i.e. it is internally hollow, and inside it the abutment element 26 is housed, which in the engagement configuration protrudes at least partly outside the relevant coupling element 8a, 8b and in the release configuration is completely enclosed inside the coupling element itself.

Appropriately, the first hooking means 6 comprise at least two abutment elements 26, which are brought nearer to each other in the release configuration and are moved away from each other in the engagement configuration.

Preferably, the abutment elements 26 are locked together in translation along the direction of mutual approach/removal 15 to the second coupling elements 8b. More particularly, the two abutment elements 26 are each housed inside a relevant second coupling element 8b.

In the release configuration, the abutment elements 26 are therefore housed inside the relevant second coupling element 8b, so that the coupling elements 8a, 8b can be positioned with their relevant supporting surfaces on the connecting frame 7, and in the engagement configuration they protrude at least partly from the second coupling elements 8b so as to place themselves below a relevant tubular element 9 of the connecting frame 7, thereby securing the load-bearing structure 2 to the connecting frame itself.

Advantageously, first control means 27 of the manual type are provided, which can be operated to move the abutment elements 26 counteracting relevant elastic means 28. Conveniently, the first control means 27 are the same for both the abutment elements 26.

Preferably, the appliance 1 also comprises second removable hooking means 29 for hooking the load-bearing structure 2 to an auxiliary structure 30, e.g. of the type of a stretcher or the like, where the second hooking means 29 can also be moved between an engagement configuration and a release configuration in order to secure and release the load-bearing structure 2 to/from the auxiliary structure 30 respectively.

More in particular, the second hooking means 29 comprise at least one gripping element 29a,29b which can be moved between the engagement configuration and the release configuration.

The second hooking means 29 also comprise second control means 12, of the type of a lever that can be manually operated by an operator, to control the displacement from the engagement configuration to the release configuration and vice versa.

The gripping element 29a, 29b can be of the rigid type, in which case it is raised and lowered in the engagement and release configuration respectively, or it can be of the type of a tensionable element, in which case it is tensioned and loosened in the engagement and release configuration, respectively.

More in detail, the gripping element 29a,29b is substantially hook shaped and its curvature is more or less arched depending on whether it is in the engagement or release configuration, respectively.

In the embodiments shown in the illustrations, the second hooking means 29 comprise two gripping elements, identified in the illustrations by the numbers 29a and 29b respectively, each of which is provided with its own relevant second control means 12.

Preferably, the second hooking means 29 comprise at least a first gripping element 29a connected to the first coupling element 8a and at least a second gripping element 29b connected to the second coupling element 8b.

Appropriately, the second gripping element 29b is locked in translation together with the second coupling element 8b along the direction of mutual approach/removal 15. The first gripping element 29a is instead associated with the first coupling element 8a and is therefore fixed with respect to the load-bearing structure 2.

In the first embodiment according to the invention, shown in Figures from 1 to 7, the first hooking means 6 and the second hooking means 29 are separate from each other.

More in detail, in the first embodiment, the first hooking means 6 and the second hooking means 29 are brought alternately to their relevant engagement and release configurations. In other words, when the load-bearing structure 2 is associated with the movement means 3, the first hooking means 6 are in the engagement configuration and the second hooking means 29 are in the release configuration, while when the load-bearing structure 2 is associated with the auxiliary structure 30, the first hooking means 6 are in the release configuration and the second hooking means 29 are in the engagement configuration.

This allows simplifying the engagement and disengagement operations, since it is necessary to adjust only the position of the second hooking means 29 for the connection to the auxiliary structure 30, the first hooking means 6 being able to engage with the connecting frame 7 regardless of the position of the second coupling element 8b.

In the second embodiment shown in the Figures from 8 to 11, the first hooking means 6 coincide with the second hooking means 29. In this embodiment, therefore, only the gripping elements 29a, 29b are provided for.

More in detail, the displacement means 16 also comprise at least one supporting element 10 associated with the connecting frame 7 in a sliding manner along the direction of mutual approach/removal 15 and the second coupling elements 8b are adapted to operate in conjunction with the supporting element 10. The supporting element 10 is locked together with the connecting frame 7 along any direction of displacement transverse to the direction of mutual approach/removal 15. The supporting element 10 therefore has only one degree of freedom with respect to the connecting frame 7.

The second gripping element 29b is therefore adapted to engage, in the engagement configuration, with the supporting element 10 in order to secure the second coupling elements 8b to the latter.

Appropriately, as in the second embodiment described above and shown in the Figures from 8 to 11, the load-bearing structure 2 comprises at least a first portion 2a and at least a second portion 2b associated with each other in a sliding manner along at least one direction of adjustment indicated in the illustrations by the double arrow 13. The direction of adjustment 13 is oriented, in use, i.e., with the movement means 3 arranged resting on the substantially vertical reference surface S.

More in particular, the first portion 2a supports at least the coupling elements 8a, 8b and the second portion 2b supports the supporting top 4; the second portion 2b can be moved in translation with respect to the first portion 2a along the direction of adjustment 13 to vary the height of the supporting top 4 with respect to the floor S.

Appropriately, the appliance 1 comprises second removable locking means 18 for locking the position of the second portion 2b with respect to the first portion 2a along the direction of adjustment 13.

More in detail, the direction of adjustment 13 is transverse to the direction of mutual approach/removal 15. Preferably, the direction of mutual approach/removal 15 is oriented perpendicularly to the direction of adjustment 13 and, in use, is arranged substantially horizontal.

In the first embodiment described above, the supporting top 4 is of the fixed type, i.e. it cannot be moved along the direction of adjustment 13. It cannot however be ruled out that the movement of the supporting top 4 along the direction of adjustment 13 can also be applied in the first embodiment.

On the supporting top 4 at least one piece of equipment can be positioned for the support of biomedical devices 20 such as, e.g., an oxygenator O and the relevant pumping unit P for extracorporeal blood circulation.

More in particular, the piece of equipment for the support of biomedical devices 20 comprises at least one holding element 21 provided with first holding means 22 for holding the oxygenator O and second holding means 23 for holding the pumping unit P which are locked together with each other.

Appropriately, the load-bearing structure 2 comprises at least one engagement element 19 to support the holding element 21 of the piece of equipment for the support of biomedical devices 20.

Preferably, the engagement element 19 is of the unified type.

Advantageously, the appliance 1 comprises removable anchoring means 31 for anchoring the biomedical device O,P which are associated with the supporting top 4.

More in particular, the anchoring means 31 comprise at least one guide element 32 associated with the supporting top 4 and extending along a direction of sliding 33 and removable locking means 34 associated with the guide element itself. The guide element 32 can be engaged in translation along the direction of sliding 33 by an anchoring element 35 associable with the biomedical device O,P and the locking means 34 are adapted to lock the anchoring element itself along the direction of sliding 33 upon reaching a predefined anchoring position. The predefined anchoring position corresponds, e.g., to the end-of-stroke position of the anchoring element 35 on the guide element 32.

The guide element 32 can be fixed to the supporting top 4 by means of the relevant fixing means 36, e.g. of the type of threaded elements, and the anchoring element 35 can, e.g., be fitted on the bottom of the piece of equipment for the support of biomedical devices 20.

Appropriately, the guide element 32 defines at least one anchoring surface 32a, arranged substantially facing the supporting top 4 and adapted, in use (i.e. when engaged with the anchoring element 35), to prevent the anchoring element 35 from lifting with respect to the guide element itself preferably, the guide element 32 defines at least one substantially C-shaped guide seat 37 intended to receive the anchoring element 35, where the anchoring surface 32a delimits such guide seat 37 above.

Advantageously, the locking means 34 comprise at least one locking element 38 movable between a locking position, wherein it is adapted to interact with the anchoring element 35 to prevent it from sliding away from the predefined anchoring position, and an unlocking position, wherein it allows the displacement of the anchoring element 35 along the direction of sliding 33. The locking means 34 also comprise elastic means 39 adapted to counteract the displacement of the locking element 38 from the locking position towards the unlocking position.

The locking element 38 has at least one locking surface 38a adapted to interact with the anchoring element 35, with the latter in the predefined anchoring position, to prevent the displacement thereof along the direction of sliding 33. The locking surface 38a is arranged transversely, and more in particular perpendicularly, to the direction of sliding 33.

The locking element 38 also has a contact surface 38b, arranged transversely with respect to the direction of sliding 33, which by interaction with the anchoring element 35 during its displacement nearer towards the predefined anchoring position permits the displacement of the locking element itself from the locking position to the unlocking position.

Preferably, the locking means 34 comprise at least two locking elements 38, between which the elastic means 39 are interposed, and which are moved away from each other in the locking position with respect to the unlocking position. The contact surfaces 38b of the locking elements 38 are arranged diverging from each other as they approach the predefined anchoring position. Each of the locking elements 38 is adapted to interact with a relevant anchoring element 35. The operation of the invention is as follows.

The appliance 1 initially has the load-bearing structure 2 secured to the movement means 3 by means of the first hooking means 6.

In this configuration of use, the load-bearing structure 2 can be moved on the reference surface S by means of the wheels 3 so as to allow the displacement of the piece of equipment for the support of biomedical devices 20 to support the biomedical devices O, P.

The piece of equipment for the support of biomedical devices 20 can be arranged on the supporting top 4 or the relevant holding element 21 can be fitted to the engagement element 19 according to the specific needs of the case.

If the piece of equipment for the support of biomedical devices 20 is arranged on the supporting top 4, it is fixed to the latter by means of the anchoring means 31. More in detail, once the anchoring elements 35 fitted to the bottom of the piece of equipment for the support of biomedical devices 20 have been aligned with the guide element 32, the anchoring elements themselves are moved forward along the direction of sliding 33. The anchoring elements 35 therefore interact with the locking elements 38, and in particular with the relevant contact surfaces 38b, displacing them from the locking position to the unlocking position. Once the anchoring elements 35 reach the predefined anchoring position, the locking elements 38 automatically displace, as a result of the action of the elastic means 39, to the locking position, where the relevant locking surface 38a interacts with the corresponding anchoring element 35, thus preventing it from moving along the direction of sliding 33. At the same time, the anchoring surface 32a prevents the anchoring elements 35 from lifting.

In the event that the biomedical devices O, P have to be removed or moved with respect to the load-bearing structure 2, the locking elements 38 are manually moved from the locking position to the unlocking position so that the anchoring elements 35 can be moved along the direction of sliding 33 away from the predefined anchoring position.

When it is necessary to move the patient, e.g., on a stretcher to transport him/her inside or outside the hospital, and therefore the biomedical devices O, P have to be adequately moved, the load-bearing structure 2 is separated from the movement means 3, bringing the first hooking means 6 to the release configuration.

In particular, in the first embodiment described above, the adjustment is made on the abutment elements 26, bringing them to the relevant release configuration so as to release them from the connecting frame 7, so as to be able to move the load-bearing structure 2 away from the movement means 3. The gripping elements 29a, 29b are, in this phase, in the release configuration.

In the second embodiment, wherein the first hooking means 6 and the second hooking means 29 coincide and comprise only the gripping elements 29a, 29b, adjustment is instead made on the second control means 12 in order to bring the gripping elements themselves to the release configuration and the load-bearing structure 2 is moved away from the movement means 3.

The load-bearing structure 2, with the relevant supporting top 4, can now be positioned on a stretcher 30 to allow the movement of the biomedical devices O, P together with the patient. In this case, since the dimensions of the stretchers are not all the same, it is possible to adjust the mutual distance of the coupling elements 8a, 8b by adjusting the displacement means 16, so as to bring the second coupling elements 8b closer or move them further away from the first coupling elements 8a. Once the desired distance has been reached, the position of the second coupling elements 8b is blocked with respect to the guide elements 17 by adjusting the first locking means 14.

Both in the first embodiment and in the second embodiment, the fixing of the load-bearing structure 2 to the stretcher 30 is carried out by adjusting the gripping elements 29a,29b.

Depending on the desired height of use of the biomedical devices O,P, the operator adjusts the first locking means 14 to vary the height of the supporting top 4 with respect to the reference surface S by moving the second portion 2b with respect to the first portion 2a along the direction of adjustment 13.

It has, in practice, been ascertained that the described invention achieves the intended objects and in particular the fact is underlined that the appliance to which the present invention relates can be used both resting on the ground, allowing the movement of the transported biomedical devices, and fitted on a stretcher or a hospital bed thanks to the possibility of separating the load-bearing structure from the movement means.

Furthermore, the appliance according to the invention can also be adapted to stretchers or beds of various types and sizes thanks to the possibility of adjusting the mutual distance of the coupling elements.

1) An appliance for the transport of biomedical devices, the appliance comprising:
at least one load-bearing structure;
movement means of said load-bearing structure with respect to a reference surface; and
at least one supporting top of at least one biomedical device associated with said load-bearing structure, wherein said load-bearing structure comprises first removable hooking means to said movement means, said first hooking means being movable between an engagement configuration, wherein said load-bearing structure is constrained to said movement means, and a release configuration wherein said load-bearing structure is released and removable from said movement means.

2) The appliance according to claim 1, wherein said movement means comprise a plurality of wheels movable in rotation around respective axes and at least one connecting frame supporting said wheels and that said load-bearing structure comprises one or more coupling elements adapted to operate in conjunction with said connecting frame, said first hooking means constraining and releasing said coupling elements to and from said connecting frame in the engagement configuration and in the release configuration, respectively.

3) The appliance according to claim 2, wherein said first hooking means comprise at least one abutment element associated with at least one of said coupling elements movable with respect thereto to switch from the engagement configuration to the release configuration and vice versa.

4) The appliance according to claim 3, wherein at least one of said coupling elements is internally hollow and, inside it, is housed said abutment element, which in the engagement configuration protrudes at least partly outside said coupling element and in the release configuration is completely enclosed inside the coupling element itself.

5) The appliance according to claim 4, further comprising:
at least two of said abutment elements, each of which associated with a relevant coupling element and wherein said abutment elements are moved close to each other in the release configuration and are moved away from each other in the engagement configuration.

6) The appliance according to ene claim 5, wherein said load-bearing structure comprises at least a first coupling element and at least a second said coupling element arranged facing each other and movable in the direction of mutual approach/removal.

7) The appliance according to claim 6, further comprising:
displacement means of said second coupling element along said direction of mutual approach/removal.

8) The appliance according to claim 7, wherein said displacement means comprise at least one guide element associated with said load-bearing structure and extending along said direction of mutual approach/removal wherein second coupling element associated in a sliding manner with said guide element.

9) The appliance according to claim 8, further comprising:
second locking means for locking the displacement of said second coupling element along said direction of mutual approach/removal.

10) The appliance according to claim 9, wherein said first coupling element is fixed with respect to said guide element.

11) The appliance according to claim 10, wherein said abutment elements are locked together in translation along said direction of mutual approach/removal to said second coupling element.

12) The appliance according to claim 11, further comprising:
second removable hooking means for hooking said load-bearing structure an auxiliary structure, said second hooking means being movable between an engagement configuration and a release configuration in order to secure and release said load-bearing structure to/from said auxiliary structure respectively.

13) The appliance according to claim 12, wherein said second hooking means comprise at least one gripping element which is raised and lowered in the engagement configuration and in the release configuration, respectively.

14) The appliance according to claim 13, wherein second hooking means comprise at least a first said gripping element connected to said first coupling element and at least a second said gripping element connected to said second coupling element.

15) The appliance according to one claim 14, wherein said second gripping element is locked in translation along said direction of mutual approach/removal together with said second coupling element.

16) The appliance according to claim 15, wherein said first hooking means coincide with said second hooking means.

17) The appliance according to claim 16, wherein said load-bearing structure comprises at least a first portion and at least a second portion associated with each other in a sliding manner along at least one direction of adjustment.

18) The appliance according to claim 17, wherein said first portion supports at least said coupling elements and that said second portion supports at least said supporting top, said second portion being associated movable in translation with said first portion along said direction of adjustment to vary the height of said supporting top with respect to the reference surface.

19) The appliance according to claim 18, further comprising: first removable locking means for locking the position of said second portion with respect to said first portion along said direction of adjustment.

20) The appliance according to claim 19, wherein said direction of adjustment is transverse to said direction of mutual approach/removal.

21) The appliance according to claim 20, further comprising: removable anchoring means for anchoring said biomedical device which are associated at least with said supporting top.

22) The appliance according to claim 21, wherein said anchoring means comprise at least one guide element associated with said supporting top and extending along a direction of sliding and removable locking means associated with said guide element, wherein said guide element can be engaged in translation along said direction of sliding by at least one anchoring element associable with said biomedical device, said locking means locking said anchoring element with respect to said guide element along said direction of sliding upon reaching a predefined anchoring position.

23) The appliance according to claim 22, wherein said guide element defines at least one anchoring surface arranged substantially facing said supporting top and adapted, in use, to prevent said anchoring element from lifting with respect to said guide element.

24) The appliance according to claim 23, wherein said guide element defines a substantially C-shaped guide seat, intended to receive said anchoring element, said anchoring surface delimiting said guide seat above.

25) The appliance according to claim 24, wherein said locking means comprise at least one locking element movable between a locking position, wherein it is adapted to interact with said anchoring element and an unlocking position, wherein the displacement of said anchoring element is allowed along said direction of sliding, and elastic means adapted to counteract the displacement of said locking element from the locking position towards the unlocking position.

26) The appliance according to claim 25, wherein said locking element is adapted to displace from the locking position to the unlocking position as a result of the sliding action of said anchoring element towards said predefined anchoring position and is adapted to displace, as a result of the action of said elastic means from the unlocking position to the locking position, upon reaching said predefined anchoring position.

27) The appliance according to claim 26, wherein said locking element has at least one locking surface adapted to interact with said anchoring element in the predefined anchoring position, to prevent the displacement thereof along said direction of sliding and at least one contact surface arranged inclined with respect to said direction of sliding and adapted to interact with said anchoring element during its displacement nearer to the predefined anchoring position, said locking element moving from the locking position to the unlocking position as a result of the interaction of said anchoring element with said contact surface.

28) The appliance according to claim 27, wherein said locking means comprise at least two of said locking elements, which are moved away from each other in the locking position with respect to the unlocking position.

29) The appliance according to claim 28, wherein the contact surfaces of said locking elements are arranged diverging outwards from each other as they approach the predefined anchoring position.

30) The appliance according to claim 29, wherein said load-bearing structure comprises at least one engagement element for the support of at least one biomedical device.
